SEQUENCE LISTING (38 sequences)

SEQ ID NO: 1 - DNA, 26 bp, Artificial Sequence
  Description of Artificial SequenceSynthetic DNA
gcccagacatatccctgaatgatacc

SEQ ID NO: 2 - DNA, 1431 bp, Saccharomyces cerevisiae
  CDS (1)..(1428)
atgaaaaaacaaaatttaaattctattttattaatgtatattaattatattattaattattttaataatattcataaaaatcaattaaaaaaagactggattatggaatatgaatatatgtataaatttttaatgaataatatgacttgttttattaaatgggataataataaaattttattattattagatatgtattataatgtattatataactatcataaacaacgtacacctatgtctaataaaagattaatgaattcaaaaaatattatggattataaattattatatacttatttttatattttaaataaaatgaaaatggaaatggataattataataataataataataatatttcattaaaatataatgaattattaaaaaatattatgaataatttaaattataaaacttctaatattgaaactaatttatctaataatttttatttaatggataaatatttaattaataaatatatgaaatatttagatatgttaaatatgattcctaataattatatgtttaataatattaattataaaggtaaattaaatattaaaacagtattagatttaaataataatgaattttatgattatttatcagggttaattgaaggtgatggttatattggtcctggaggtattacaattactaatcatgctaatgatgtattaaatactatctttattaataaaagaattaaaaatagtattttagtagaaaaatggatggatactttaaaagataatccttattttgttaatgctttctctattaatattaaaactaatttagctaaagaaaagatttttactaatatttataataaattatatagtgattataaaattaatcaaattaataatcatatcccttattataattatttaaaaattaataataaattacctattaaaaatattatggatattaaaaataattattggttagctggttttacagctgcagatggttcttttttatcatctatgtataatcctaaagatacattattatttaaaaatatgagacctagttatgttatttcacaagttgaaacacgtaaagaattaatttatttaattcaagaatcttttgatttatctatttctaatgttaaaaaagttggtaatagaaaattaaaagattttaaattatttaccagaactactgatgaattaatgaaatttatttattattttgataaatttttacctttacatgataataaacaatttaattatattaaatttagatttaatacttttattaaatcatataattggaataatagagtatttggtttagtattatctgaatatatcaataatattaaaattgataattatgattattattattataataaatatattaatatgcataatgcacgtaaacctaaaggatacattaaataa

SEQ ID NO: 3 - protein, 476 aa, Saccharomyces cerevisiae
MKKQNLNSILLMYINYIINYFNNIHKNQLKKDWIMEYEYMYKFLMNNMTCFIKWDNNKILLLLDMYYNVLYNYHKQRTPMSNKRLMNSKNIMDYKLLYTYFYILNKMKMEMDNYNNNNNNISLKYNELLKNIMNNLNYKTSNIETNLSNNFYLMDKYLINKYMKYLDMLNMIPNNYMFNNINYKGKLNIKTVLDLNNNEFYDYLSGLIEGDGYIGPGGITITNHANDVLNTIFINKRIKNSILVEKWMDTLKDNPYFVNAFSINIKTNLAKEKIFTNIYNKLYSDYKINQINNHIPYYNYLKINNKLPIKNIMDIKNNYWLAGFTAADGSFLSSMYNPKDTLLFKNMRPSYVISQVETRKELIYLIQESFDLSISNVKKVGNRKLKDFKLFTRTTDELMKFIYYFDKFLPLHDNKQFNYIKFRFNTFIKSYNWNNRVFGLVLSEYINNIKIDNYDYYYYNKYINMHNARKPKGYIK

SEQ ID NO: 4 - DNA, 18 bp, Artificial Sequence
  Description of Artificial SequenceSynthetic DNA
ccggatccatgaaaaaac

SEQ ID NO: 5 - DNA, 23 bp, Artificial Sequence
  Description of Artificial SequenceSynthetic DNA
gggtcgacttatttaatgtatcc

SEQ ID NO: 6 - DNA, 19 bp, Artificial Sequence
  Description of Artificial SequenceSynthetic DNA
aaaagactggattatagaa

SEQ ID NO: 7 - DNA, 19 bp, Artificial Sequence
  Description of Artificial SequenceSynthetic DNA
tgaatatatgtataaattt

SEQ ID NO: 8 - DNA, 19 bp, Artificial Sequence
  Description of Artificial SequenceSynthetic DNA
tattaaatgggataataat

SEQ ID NO: 9 - DNA, 21 bp, Artificial Sequence
  Description of Artificial SequenceSynthetic DNA
tattagatatgtattataatg

SEQ ID NO: 10 - DNA, 19 bp, Artificial Sequence
  Description of Artificial SequenceSynthetic DNA
tacacctatgtctaataaa

SEQ ID NO: 11 - DNA, 20 bp, Artificial Sequence
  Description of Artificial SequenceSynthetic DNA
aaaatattatggattataaa

SEQ ID NO: 12 - DNA, 52 bp, Artificial Sequence
  Description of Artificial SequenceSynthetic DNA
ttttatattttaaataaaatgaaaatggaaatggataattataataataata

SEQ ID NO: 13 - DNA, 21 bp, Artificial Sequence
  Description of Artificial SequenceSynthetic DNA
aaaatattatgaataatttaa

SEQ ID NO: 14 - DNA, 36 bp, Artificial Sequence
  Description of Artificial SequenceSynthetic DNA
actatctaatattgaaactaatttatctaataattt

SEQ ID NO: 15 - DNA, 19 bp, Artificial Sequence
  Description of Artificial SequenceSynthetic DNA
ttatttaatggataaatat

SEQ ID NO: 16 - DNA, 21 bp, Artificial Sequence
  Description of Artificial SequenceSynthetic DNA
ataaatatatgaaatatttag

SEQ ID NO: 17 - DNA, 21 bp, Artificial Sequence
  Description of Artificial SequenceSynthetic DNA
ataattatatgtttaataata

SEQ ID NO: 18 - DNA, 33 bp, Artificial Sequence
  Description of Artificial SequenceSynthetic DNA
ggaggtattacaattactaatcatgctaatgat

SEQ ID NO: 19 - DNA, 36 bp, Artificial Sequence
  Description of Artificial SequenceSynthetic DNA
ttttagtagaaaaatggatggatactttaaaagata

SEQ ID NO: 20 - DNA, 39 bp, Artificial Sequence
  Description of Artificial SequenceSynthetic DNA
agctaaagaaaagatttttactaatatttataataatta

SEQ ID NO: 21 - DNA, 19 bp, Artificial Sequence
  Description of Artificial SequenceSynthetic DNA
aaatattatggatattaaa

SEQ ID NO: 22 - DNA, 18 bp, Artificial Sequence
  Description of Artificial SequenceSynthetic DNA
taattattggttatctgg

SEQ ID NO: 23 - DNA, 19 bp, Artificial Sequence
  Description of Artificial SequenceSynthetic DNA
atcatctatgtataatcct

SEQ ID NO: 24 - DNA, 19 bp, Artificial Sequence
  Description of Artificial SequenceSynthetic DNA
ttaaaaatatgagacctag

SEQ ID NO: 25 - DNA, 23 bp, Artificial Sequence
  Description of Artificial SequenceSynthetic DNA
gatgaattaatgaaatttattta

SEQ ID NO: 26 - DNA, 39 bp, Artificial Sequence
  Description of Artificial SequenceSynthetic DNA
attaaatttagatttaatacttttattaaatcatataat

SEQ ID NO: 27 - DNA, 38 bp, Artificial Sequence
  Description of Artificial SequenceSynthetic DNA
tataataaatatattaatatgcataatgcacgtaaacc

SEQ ID NO: 28 - DNA, 42 bp, Artificial Sequence
  Description of Artificial SequenceSynthetic DNA
taaatttttaataaataatatgacttgttttattaaatggga

SEQ ID NO: 29 - DNA, 19 bp, Artificial Sequence
  Description of Artificial SequenceSynthetic DNA
aagattaatgaattcaaaa

SEQ ID NO: 30 - DNA, 39 bp, Artificial Sequence
  Description of Artificial SequenceSynthetic DNA
gattataaattattatatacttatttttatattttaaat

SEQ ID NO: 31 - DNA, 38 bp, Artificial Sequence
  Description of Artificial SequenceSynthetic DNA
gaataatttaaattataaaacttctaatattgaaacta

SEQ ID NO: 32 - DNA, 37 bp, Artificial Sequence
  Description of Artificial SequenceSynthetic DNA
ttctctattaatattaaaactaatttagctaaagaaa

SEQ ID NO: 33 - DNA, 38 bp, Artificial Sequence
  Description of Artificial SequenceSynthetic DNA
aaattatttaccagaactactgatgaattaatgaaatt

SEQ ID NO: 34 - DNA, 20 bp, Artificial Sequence
  Description of Artificial SequenceSynthetic DNA
catataattggaataataga

SEQ ID NO: 35 - DNA, 20 bp, Artificial Sequence
  Description of Artificial SequenceSynthetic DNA
aatttttaatgaataatatg

SEQ ID NO: 36 - DNA, 19 bp, Artificial Sequence
  Description of Artificial SequenceSynthetic DNA
tttagatatgttaaatatg

SEQ ID NO: 37 - DNA, 25 bp, Artificial Sequence
  Description of Artificial SequenceSynthetic DNA
atatgttaaatatgattcctaataa

SEQ ID NO: 38 - DNA, 20 bp, Artificial Sequence
  Description of Artificial SequenceSynthetic DNA
ctggattatggaatatgaat

(19) United States
(12) Patent Application Publication
(10) Pub. No.: US 2003/0215439 A1
(43) Pub. Date: Nov. 20, 2003

(54) Therapeutic uses of PAF-AH products in diabetes

(76) Inventors: Gregory N. Dietsch, Snohomish, WA (US); Gary M. Peterman, Seattle, WA (US); Albert S. Yu, Bothell, WA (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/373,639

(22) Filed: Feb. 24, 2003

Related U.S. Application Data
(63) Continuation of application No. 09/774,414, filed on Jan. 31, 2001, now abandoned.
Application No. 09/774,414 is a continuation of application No. 09/306,970, filed on May 7, 1999, now abandoned.

(51) Int. Cl. A61K 38/48

(52) U.S. Cl. 424/94.63

(57) ABSTRACT

The present invention relates to uses of PAF-AH products to prevent or slow the progression of diabetes, particularly insulin dependent diabetes mellitus.

### FIELD OF THE INVENTION

The present invention relates generally to novel therapeutic methods for preventing the progression of diabetes mellitus by administration of platelet activating factor acetylhydrolase (PAF-AH) products.

### BACKGROUND OF THE INVENTION

Insulin-dependent diabetes mellitus, or IDDM, is a disease of metabolic dysregulation, particularly glucose metabolism dysregulation, with severe and long-term health consequences for sufferers, including vascular and neurologic complications. The predominant abnormality in patients with IDDM is a deficiency in insulin, the major anabolic hormone in humans.

IDDM, also known as “Type I” or juvenile onset diabetes, affects 1 in 250 Americans, with 10,000-15,000 new cases every year. Its prevalence is greatest in Caucasians, its frequency in that population being twice that among people of African and Asian ancestry. Typically, clinical onset is during childhood. The majority of sufferers are diagnosed before the age of 20, with peak onset around puberty; fewer than 10% of cases first appear in patients over the age of 50. The survival of patients suffering from IDDM depends entirely on the intake of exogenous insulin.

The acute clinical onset of IDDM is characterized by symptoms of hyperglycemia (polyuria, polydipsia, weight loss, or blurred vision, alone or in combination), followed days or weeks later by ketoacidosis or diagnosis. It is now accepted that the acute onset of the disease is preceded by a long, asymptomatic preclinical period, during which the insulin-secreting β-cells are progressively destroyed. In healthy individuals, the pancreas normally contains 1 to 1.5 million islets; approximately 80 percent of islet cells are insulin-producing β-cells. The symptoms of clinical diabetes appear when fewer than 10 percent of those β-cells remain.

The progressive destruction of the body's ability to regulate glucose metabolism is believed to be caused by insulitis, or lymphocytic infiltration of the islets, with concomitant changes in T cell subpopulations, such as increased suppressor-inducer T cells and decreased helper-inducer T cells. The appearance of islet cell antibodies (ICAs) and other antibodies up to 10 years prior to clinical symptoms identifies those patients-in whom the inexorable destruction of insulin-secreting β-cells has begun. Genetic factors have been identified which may indicate predisposition to or protection from IDDM, and environmental factors also influence the development of the disease.

A mismatch between insulin supply and demand leads to abnormal glucose, lipid and protein metabolism. Insulin deficiency may lead to hyperglycemia and hyperglycemic dehydration, elevated levels of free fatty acids, elevated serum ketone levels, increased levels of triglycerides, very low density lipoproteins (VLDLs), and branched chain amino acids, a decrease in protein synthesis, and ketoacidosis. Persons with IDDM are likely to suffer from a variety of vascular and neurologic complications; the risk of developing macrovascular disease, including cardiac, peripheral and cerebrovascular disease, is much greater in diabetic patients than in the population at large. In general, IDDM patients are two times more likely than non-diabetics to have a heart attack; they are five times more likely to suffer from gangrene; seventeen times more likely to have complete renal failure, and twenty-five times more likely to lose their eyesight.

Complications specific to diabetes include retinopathy and nephropathy. With conventional insulin management, more than 90% of IDDM patients are diagnosed with retinopathy after 15 years' duration of IDDM. Retinopathy is characterized by microaneurysms caused by a loss of pericytes, the cells which form the support of the retinal vasculature; small hemorrhages leak blood and serum into the retina, causing the formation of hard exudates, which can lead to visual deficits. In more severe retinopathy, obstructed capillaries cause ischemic injury to the retina, leading to infarctions and the proliferation of extremely fragile blood vessels in the retina. Bleeding from these vessels can lead to retinal detachment due to scars formed upon reabsorption of the blood, leading to severe and permanent visual impairment. Laser treatment has been shown to be effective in restoring vision in patients with less severe proliferative retinopathy and macular edema.

Nephropathy is associated with the highest mortality of any of the complications of diabetes, and occurs in 35-45 percent of IDDM patients. Microalbuminuria progresses to macroalbuminuria with hypertension after 12 to 20 years of IDDM. Finally, the nephrotic syndrome and the decrease glomerular filtration rate lead to end stage renal disease. The development of diabetic nephropathy is associated with an especially high risk of coronary artery disease.

The most common diabetic neuropathy is a peripheral neuropathy that manifests as numbness or tingling in the toes or feet, which may abate over time as paresthesias and dysesthesis progress to hypoesthesia or anesthesia. Insensate feet are vulnerable to injury, and hospitalization and amputation may result from neuropathic foot ulcers. Other neuropathies associated with diabetes include mononeuropathy, entrapment syndrome, and autonomic neuropathy, some of which are treatable with varying degrees of success. Insulin deficiency is thought to lie at the root of all of these complications.

The maintenance of normoglycemia is extremely difficult with even the most rigorous treatment regimen. Intensive treatment, which is required to prevent the appearance of deleterious side effects, involves monitoring blood glucose levels and multiple daily injections of insulin, under the oversight of a team of experts, necessitating huge resources and exemplary compliance on the part of the patient. Pancreas transplantation, while often successful, suffers from the same drawbacks as any organ transplantation effort: the risks of major surgery, long-term immunosuppression and its side effects, donor matching, etc. Islet transplantation may be more successful but again requires surgical intervention. Artificial pancreas technology has not yet met the challenge of automatically delivering the proper dose of insulin in response to sensed glucose levels, thus, current models require the same self-monitoring of more traditional injection therapy, and again require surgery. Immunosuppressive drugs, such as azathioprine, prednisone, and cyclosporine, have been tested for their ability to stop beta cell destruction, but long term toxicity, especially nephrotoxicity and the rapid loss of islet function following withdrawal of the drugs has made them less than ideal therapeutic candidates. [Nathan, D. M., Diabetes Mellitus, Scientific American Medicine, 9:VI: 1-24, rev. November/1997.]

Thus, there remains a need for additional agents effective for preventing or treating diabetes.

Platelet-Activating Factor (PAF) is a biologically active phospholipid synthesized by various cell types. PAF has been found to activate cells involved in inflammation, including neutrophils, eosinophils, platelets, mast cells, and macrophages. [Venable et al. (1993), supra.] Its receptor (PAF-R) is expressed on endothelial cells, neutrophils, monocytes, macrophages, and platelets. In vivo and at normal concentrations of 10⁻¹⁰ to 10⁻⁹ M, PAF activates target cells such as platelets and neutrophils by binding to specific G protein-coupled cell surface receptors (herein designated PAF-R). [Venable et al., J Lipid Res 34:691-703, 1993.] PAF has the structure 1-Ω-alkyl-2-acetyl-sn-glycero-3-phosphocholine. For optimal biological activity, the sn-1 position of the PAF glycerol backcone must have a phosphocholine head group.

Synthesis and secretion, as well as degradation and clearance, of PAF appear to be tightly regulated. PAF can be synthesized by two different pathways: by de novo synthesis or by remodeling, with the remodeling pathway thought to be responsible for producing the majority of PAF and to be more important in various inflammatory and allergic response. [Venable et al. (1993), supra.] In the remodeling pathway, the precursor form of PAF, alkyl acyl glycerophosphocholine (GPC), is stored in the membrane of cells such as endothelial cells and is converted to biologically inactive lyso-PAF by phospholipase A₂ upon inflammation or cell injury. The subsequent transfer of an acetyl group to the SN² position (C2) of the glycerol backbone forms PAF. PAF can be converted back to the inactive lyso-PAF through deacetylation by PAF acetylhydrolase (PAF-AH), an enzyme activity released by macrophages and hepatocytes.

Two forms of PAF-AH have been identified: a cytoplasmic form, found in a variety of cell types and capable of hydrolyzing PAF as well as oxidatively fragmented phospholipids such as products of the arachidonic acid cascade that mediate inflammation [Stremler et al., J Biol Chem 266(17):11095-11103, 1991]; and an extracellular type, found in plasma and serum, which is likely to regulate inflammation. Plasma PAF-AH is specific for PAF; it does not hydrolyze other intact phospholipids. This substrate specificity allows the enzyme to circulate in vivo in a fully active form without adverse effects. The plasma PAF-AH appears to account for all of the degradation of PAF in human blood ex vivo. [Stafforini et al., J Biol Chem 262(9):4223-4230, 1987.] The plasma form of PAF-AH has been cloned and expressed as a recombinant protein (rPAF-AH). See U.S. Pat. Nos. 5,532,152 and 5,641,669, incorporated herein by reference. Pretreatment with rPAF-AH effectively blocks PAF-induced rat paw edema as well as microvascular leakage in rat pleurisy, suggesting that rPAF-AH is a potent inflammatory inhibitor in vivo. [Tjoelker et al., Nature 374:549-553 (1995).]

PAF functions in normal physiological processes (e.g. inflammation, hemostasis and parturition) and has been suggested as implicated in pathological inflammatory responses (e.g., asthma, anaphylaxis, septic shock and arthritis). [Venable et al (1993), supra; Lindsberg et al., Ann Neurol 30:117-129, 1991.] PAF-induced biological activities also have been suggested as implicated in increased vascular permeability, leukocyte adhesion to endothelial cells, and hypotension. [Venable et al. (1993), supra.] The involvement of PAF in pathological reponses has prompted attempts to modulate the activity of PAF. The major focus of these attempts until now has been the development of PAF-R antagonists, i.e., inhibitors of PAF activity which interfere with the binding of PAF to cell surface receptors, generally via competitive mechanisms.

Treatment of diabetes-prone rats from 30 days of age with ginkgolide B (BN 52021), an agent which competitively blocks the PAF-R, has been reported to reduce the severity of insulitis but did not affect the frequency or age of onset of diabetes in these rats. [Beck et al., Autoimmunity 9(3):225-35, 1991.] Administration of BN 52021 has also been reported to afford dose-dependent protection against anti-islet cell toxicity [Kohler et al., Int Arch Allergy Appl Immunol 95:352-55, 1991], but that report failed to rule out a PAF-independent action of anti-islet cell toxicity. A synthetic PAF analogue, BN 50730, has been reported to reduce insulitis and the frequency of diabetes in a dose-dependent manner. [Jobe et al., Autoimmunity 16:259-266, 1993.] SR 27388, a competitive antagonist of PAF binding to the PAF-R, has been shown to protect mice against alloxan-induced diabetes. [Herbert et al., J Lipid Mediat 8(1):31-51, 1993.] Involvement of PAF in insulitis and autoimmune β-cell destruction has been suggested. [Lee et al., Diabetes 48(1):43-9, 1999.] However, plasma levels of PAF and PAF-AH in diabetes have been the subject of some controversy. IDDM patients have been reported to have a 50-fold elevated PAF blood level compared to healthy volunteers, despite similar levels of PAF precursors and PAF-AH activity. [Nathan et al., Diabete Metab 18(1):59-62, 1992.] PAF degradation has been reported to be 17.5% higher in IDDM patients compared to matched controls. [Hofmann et al., Haemostasis 19(3):180-84, 1989.] Yet a third study reported a 35% decrease in PAF-AH activity in IDDM patients compared to healthy individuals. [Memon et al., J Pak Med Assn 45(5):122-125, 1995.] A study in the streptozotocin (STZ)-induced diabetic rat model reported no difference in PAF-AH activity between well-fed non-diabetic rats and STZ-diabetic rats. [Trapali et al., Life Sci 59(10):849-57, 1996.] A different study indicated that STZ-induced diabetic rats produced greater amounts of PAF in response to the same stimulus compared to non-diabetic rats. [Akiba et al., J Biochem 117(2):425-31, 1995.] Thus, there is little or no consensus in the literature on the involvement of PAF and PAF-AH in diabetes.

PAF has been suggested as implicated in some disorders that may be associated with diabetes, e.g., retinal ischemia [De la Cruz et al., Eur J Pharmacol 360(1):37-42, 1998], cardiovascular disease [Shukla et al., Thromb Res 66(2-3):239-46, 1992; Fritschi et al., Thromb Haemost 52(3):236-9, 1984, Juhan-Vague et al., Thromb Res 38(1):83-9, 1985], inflammatory response to ischemia-reperfusion [Salas et al., Am J Physiol 275(5 pt. 2):H1773-81, 1998] and a depressor response induced by PAF [Abiru et al., J Pharmacobiodyn 14(6):293-300, 1991].

### SUMMARY OF THE INVENTION

The present invention provides novel therapeutic uses for PAF-AH products in subjects at risk of diabetes or subjects suffering from diabetes, particularly early stage diabetes, and is based on the discovery that administration of a PAF-AH analog (rPH.2, described below) reduced the frequency of diabetes in diabetes-prone rats and preserved the function of pancreatic islet β cells that produce insulin. Thus, the invention provides methods of preventing and slowing the progression of diabetes, particularly insulin dependent diabetes mellitus (IDDM), by administering a therapeutically effective amount of a PAF-AH product.

Administration of PAF-AH products to subjects at risk of diabetes (e.g., subjects with HLA class II alleles associated with diabetes, elevated anti-islet cell antibody levels, signs of insulitis or signs of islet cell infiltration) or subjects suffering from diabetes (e.g. with impaired glucose tolerance or clinical symptoms of diabetes) is contemplated. Suitable subjects include mammals, particularly humans, or other animals. The PAF-AH product can be administered at doses ranging from about 1 μg/kg to 100 mg/kg daily, or preferably 0.5 to 50 mg/kg daily, or most preferably at a dose of 5 to 101 mg/kg over a 24 hour period, varying in children and adults. The dosage may be administered once daily, or in equivalent doses at longer or shorter intervals, for e.g. 5 days. Presently preferred PAF-AH products include fragments having Met₄₆ of SEQ ID NO: 2 as the N-terminal residue and Ile₄₂₉ or Asn₄₄₁ as the C-terminal residue.

The invention further provides use of PAF-AH products in the manufacture of a medicament for preventing or treating diabetes.

Numerous additional aspects and advantages of the invention will become apparent to those skilled in the art upon consideration of the following detailed description of the invention which describes presently preferred embodiments thereof

### BRIEF DESCRIPTION OF THE DRAWINGS

FIGS. 1A and 1B show the frequency of diabetes in diabetes prone rats treated with various doses of rPAF-AH product or control.

FIG. 2 shows the Kaplan-Meier survival analysis for diabetes prone rats treated with rPAF-AH product or control.

### DETAILED DESCRIPTION

The present invention provides novel therapeutic methods involving the administration of PAF-AH products for preventing or slowing the progression of diabetes. Unlike conventional diabetic therapies, which rely on administration of exogenous insulin or agents that stimulate insulin release from remaining responsive pancreatic islet cells, therapeutic administration of PAF-AH products is expected to be effective for preventing and/or treating the underlying disease itself PAF-AH administration may result in a reduced incidence of diabetes, a later onset of insulin dependence, maintenance of detectable C-peptide levels (a marker of endogenous insulin production), slower or arrested progression of the disease or a reduced severity of diabetes. Thus, therapeutically effective amounts of PAF-AH product include: amounts effective to prevent diabetes, including an amount effective to reduce the incidence of diabetes; and amounts effective to slow the progression of diabetes, including an amount effective to delay onset of insulin dependence, to resolve insulitis, to reduce the severity of insulitis or islet cell infiltration, to prevent or halt further destruction of pancreatic islet cells, to prevent a substantial reduction of or slow the rate of reduction of C-peptide levels, or to reduce the severity of diabetes (as indicated, for example, by an increase in the relative number of functioning pancreatic islet β cells, or by relatively increased levels of circulating insulin either during fasting or in response to administration of glucose, or by reduced insulin requirements, or by closer-to-normal blood glucose levels following a glucose challenge, i.e., a glucose tolerance test). PAF-AH products may not only slow the progression of or reduce the severity of diabetes, but may also arrest or reverse the progression of the disease.

Subjects that may be treated according to the methods of the invention include subjects with diabetes (including IDDM and adult onset diabetes), particularly early stage diabetes, and subjects at risk of diabetes. Subjects at risk of diabetes include subjects that have genetic factors indicating a predisposition to diabetes (e.g., the presence or absence of certain alleles, particularly at the DQ and DR loci of the class II major histocompatibility complex (MHC), has been strongly associated with the development of IDDM [Gottlieb et al., Ann. Rev. Med. 49:391-405 (1998), incorporated herein by reference]); subjects with immunologic evidence of anti-islet cell abnormalities (e.g., elevated levels of anti-islet cell antibodies) that indicate a risk of diabetes or early onset of diabetes [Ziegler et al., Diabetes Care, 13(7):762-5 (1990), incorporated herein by reference]; subjects with signs of early insulitis or lymphocytic infiltration of the islets, with concomitant changes in T cell subpopulations, such as increased suppressor-inducer T cells and decreased helper-inducer T cells; and subjects with early signs of pre-diabetic abnormalities, such as an abnormal IV glucose tolerance test. For example, the presence of HLA class II allele DQbeta1*0302 (in Caucasians) or HLA class II allele DQbeta1*0201 (in non-Caucasians) have been strongly associated with IDDM, while the absence of HLA class II allele DQbeta1*0602 (a marker for protection against development of diabetes) has been strongly associated with IDDM. In addition, elevated levels of antibodies to islet beta-cell proteins, termed “anti-islet cell antibodies” herein, such as anti-insulin antibodies, anti-GAD (glutamic acid decarboxylase) antibodies and anti-islet antigen₂ antibodies are a marker for eventual onset of diabetes. The presence of IDDM-associated alleles and two out of three of the antibody markers has been correlated with a 90% probability of developing clinical diabetes mellitus within 5 years.

The PAF-AH product can be administered to subjects at doses ranging from about 1 μg/kg to 100 mg/kg daily, or preferably 0.5 to 50 mg/kg daily or most preferably 5 to 10 mg/kg daily.

The drug may be administered systemically or topically. Systemic routes include e.g., oral, intravenous, intramuscular or subcutaneous injection (including into depots for long-term release), or intraocular, retrobulbar, intraventricular, intrathecal (into cerebrospinal fluid), intraperitoneal, intrapulmonary or transdermal routes. The drug may be aerosolized for pulmonary administration or formulated in a spray for nasal administration. Topical routes include administration in the form of salves, ointments, creams, jellies, patches, ophthalmic drops or opthalmic ointments, ear drops, vaginal or rectal suppositories, enemas, or in irrigation fluids (for, e.g., irrigation of wounds).

The drug may be administered parenterally via continuous intravenous infusion, via periodic brief intravenous infusions, or by bolus. Smaller doses can be used at shorter intervals, e.g., multiple times daily, or equivalent dosing of PAF-AH products with a longer half-life can be accomplished at longer intervals. The therapeutically effective dose may be adjusted to provide maximum clinical benefit without resulting in excessive toxicity.

The dosage of the drug may be increased or decreased, and the duration of treatment may be shortened or lengthened as determined by the treating physician. The frequency of dosing will depend on the pharmacokinetic parameters of the agents and the route of administration. The optimal pharmaceutical formulation will be determined by one skilled in the art depending upon the route of administration and desired dosage. See for example, Remington's Pharmaceutical Sciences, 18th Ed. (1990, Mack Publishing Co., Easton, Pa. 18042) pages 1435-1712, the disclosure of which is hereby incorporated by reference. Such formulations may influence the physical state, stability, rate of in vivo release, and rate of in vivo clearance of the administered agents.

Those of ordinary skill in the art will readily optimize effective dosages and administration regimens as determined by good medical practice and the clinical condition of the individual patient. Regardless of the manner of administration, the specific dose may be calculated according to body weight, body surface area or organ size. Further refinement of the calculations necessary to determine the appropriate dosage for treatment involving each of the above mentioned formulations is routinely made by those of ordinary skill in the art without undue experimentation, especially in light of the dosage information and assays disclosed herein, as well as the pharmacokinetic data observed in human clinical trials. Appropriate dosages may be ascertained through use of established assays for determining blood levels dosages in conjunction with appropriate dose-response data. The final dosage regimen will be determined by the attending physician, considering various factors which modify the action of drugs, e.g. the drug's specific activity, the severity of the damage and the responsiveness of the patient, the age, condition, body weight, sex and diet of the patient, the severity of any infection, time of administration and other clinical factors. As studies are conducted, further information will emerge regarding the appropriate dosage levels for the treatment of various diseases and conditions.

Co-administration of PAF-AH products with other agents that treat diabetes or symptoms of diabetes (e.g., insulin or sulfonylureas) is also contemplated. If the second agent is a PAF inhibitor or antagonist, the dosage of each agent required to exert a therapeutic effect during combinative therapy may be less than the dosage necessary for monotherapeutic effectiveness. Treatment with PAF-AH products according to the present invention may also provide an added clinical benefit by reducing the severity of complications, e.g., cardiovascular disease, retinopathy, nephropathy and neuropathy associated with diabetes.

The term “PAF-AH products” as used herein includes natural, recombinantly produced or synthetic human PAF-AH and fragments, variants and derivatives thereof that retain biological activity of PAF-AH according to the methods of the invention. The nucleotide and amino acid sequences of human PAF-AH are set forth in SEQ ID NOS: 1 and 2, respectively, and are also found in e.g., U.S. Pat. Nos. 5,532,152 and 5,641,669, incorporated herein by reference. Variants may comprise PAF-AH analogs wherein one or more of the specified (i.e., naturally encoded) amino acids is deleted or replaced or wherein one or more nonspecified amino acids are added, without loss of the biological PAF-AH activity that mediates prevention of or retards progression of diabetes. PAF-AH variants include fusion proteins in which PAF-AH or PAF-AH fragments or PAF-AH analogs are fused to, e.g., targeting agents (such as monoclonal antibodies specific for pancreatic cells) or agents that improve half-life (such as immunoglobulin constant regions). Derivatives of PAF-AH polypeptides, PAF-AH fragments or PAF-AH variants include derivatized polypeptide containing water soluble polymers, such as polyethylene glycol. A variety of PAF-AH products are disclosed in allowed, co-owned, co-pending U.S. Ser. No. 08/910,041 filed Aug. 12, 1997, incorporated herein by reference, and use of any of these PAF-AH products according to the methods of the invention is contemplated.

Examples of PAF-AH fragments include fragments lacking up to the first twelve N-terminal amino acids of the mature human PAF-AH amino acid sequence set out in SEQ ID NO: 2, particularly those having Met₄₆, Ala₄₇ or Ala₄₈ of SEQ ID NO: 2 as the initial N-terminal amino acid. Also contemplated are fragments thereof lacking up to thirty C-terminal amino acids of the amino acid sequence of SEQ ID NO: 2, particularly those having Ile₄₂₉ and Leu₄₃, as the C-terminal residue.

Examples of PAF-AH variants include: full length human PAF-AH or fragments having amino acid substitutions in the sequence of SEQ ID NO: 2 selected from the group consisting of S 108 A, S 273 A, D 286 A, D 286 N, D 296 A, D 304 A, D 338 A, H 351 A, H 395 A, H 399 A, C 67 S, C 229 S, C 291 S, C 334 S, C 407 S, D 286 A, D 286 N and D 304 A.

Presently preferred PAF-AH products include the polypeptide expression products of DNA encoding amino acid residues Met₄₆ through Asn₄₄₁ of SEQ ID NO: 1, designated rPH.2, and the polypeptide expression products of DNA encoding amino acid residues Met₄₆ through Ile₄₂₉ of SEQ ID NO: 1, designated rPH.9.

Other aspects and advantages of the present invention will be understood upon consideration of the following illustrative examples. Example 1 addresses the effect of a rPAF-AH product, rPH.2, in a diabetic rat model.

### EXAMPLE 1

### Effect of a rPAF-AH Product in a Diabetic Rat Model

The effect of a rPAF-AH product, rPH.2, in a diabetic rat model was evaluated as follows. A total of 115 male BB/Worcester (BB/Wor) rats from the inbred diabetes prone (DP) BF subline (University of Massachusetts, Worcester, Mass.) were received at 24-25 days of age and allowed to acclimatize until 35 days of age. These BF rats have been reported to have a frequency of diabetes exceeding 86% by 100 days of age, with a mean age of onset of 80 days. [Like et al., Diabetes, 40:259-262, 1991] The rats were randomly distributed in each of the two series of experiments A and B.

In experiment A, 45 rats were randomized into three groups (n=15 each). From age 35 days until either the onset of diabetes or until the rats reached age 120 days, each group was given daily i.p. injections of blinded treatments of either 1.0 mg/kg body weight of rPH.2, 0.5 mg/kg rPH.2, or 1.0 mg/kg inactivated rPH.2 [inactivated with p-aminoethyl benzenesulfonyl fluoride (PEFABLOC®; Sigma Chemical Co., St. Louis, USA) at a ratio of 0.0265:1 and tested by PAF-AH activity assay]. As a baseline group, another 5 rats were left untreated to be euthanized at 60 days of age. In experiment B, 60 rats were divided into 2 groups (n=30 each) and, starting at age 35 days until onset of diabetes or age 120 days, were given daily i.p. injections of either 6.0 mg/kg body weight rPH.2 or vehicle as a control. Another 5 rats were left untreated until euthanized at 60 days of age.

The rats were fed a regular diet and were kept in SPF conditions with a standard light cycle. Except for the rats killed after the onset of diabetes, all animals survived for the entire 120 days. Blood glucose was measured by a Glucometer® (MediSense Inc., Waltham, Mass.) if the daily monitored body weight decreased. In experiment A, diabetes onset was diagnosed by a blood glucose level of >240 mg/dl (12 mM) for 2 consecutive days. In experiment B, diabetes onset was diagnosed by two high blood glucose readings of >240 mg/dl on the same day (once in the morning and once in the afternoon) so that samples could be collected from the rats on the first day of diagnosis.

In experiment A, without the morning injection of rPH.2, rats were euthanized either when diagnosed with diabetes or at 120 days of age. The pancreas, thyroid, spleen, thymus, adrenal gland, liver, and kidney were removed and weighed. In experiment B, the rats were euthanized and dissected within 8 hours of the morning rPH.2 injection so that serum PAF-AH activity levels could be measured to evaluate the effectiveness of i.p. administration of rPAF-AH product.

In both experiments A and B, the removed pancreases were fixed in 4% paraformaldehyde and embedded in paraffin. Sections (Ø8 μm) were stained with hematoxylin and eosin and evaluated by light microscopy for signs of insulitis, by more than four independent investigators blinded as to treatment. The degree of insulitis was the average of the scores on coded duplicate slides according to the following scale: 0, normal islets with no mononuclear cells infiltration, +1, infiltration in the islet periphery but core and mantle still identifiable; +2, mixed islet appearances, varying from unaffected islets to end-stage, with overall mild inflammation; +3, infiltration into the islet core with some β-cell remnants; +4, end-stage islets with core filled with infiltrates, with no recognizable β cells. Thyroid tissue from experiment A was treated similarly and scored according to the following scale for thyroiditis: 0, normal core, with no mononuclear cells infiltration; +1, foci of lymphocytic infiltration; +2, infiltration in and around the follicles, showing elongation of the thyrocytes and loss of colloid.

In both experiments A and B, multiple pancreas sections were examined for insulin (β cells) and glucagon (a cells). Slides were stained with anti-glucagon and anti-insulin antibodies as follows and randomly coded. Polyclonal rabbit anti-rat glucagon antibodies (Dr. D. Baskin, University of Washington, Seattle) diluted 1:200 in PBS with 2% goat serum and 1% BSA were added to deparaffined, rehydrated sections and incubated for 1 hour at room temperature. Biotinylated anti-rabbit IgG (H+L) (Vector Laboratories, Burlingame, Calif.) and alkaline phosphatase streptavidin (Vector Laboratories) were used to detect specific binding of the anti-glucagon antibodies. 4-Nitro blue tetrazolium chloride/5-Bromo-4-chloro-3-indolyl-phosphate (NBT/Br-X-Phosphate, Boehringer Mannheim, Indianapolis, Ind.) was used as a substrate for the alkaline phosphatase. The slides were then treated with monoclonal guinea pig anti-human insulin antibodies diluted 1:50 in PBS with 2% goat serum and 1% BSA (BioGenex, San Ramon, Calif.), followed by an overnight incubation at 4 C. Subsequent applications of biotinylated anti-guinea pig IgG (H+L) (Vector Laboratories), alkaline phosphatase streptavidin (Vector Laboratories) and “Fast Red” alkaline phosphatase substrate (Vector Laboratories) were applies to detect the binding of the anti-insulin antibodies. The double-stained slides were counter-stained with methyl green, dehydrated, and mounted.

Images from the sections were retrieved with a Nikon Optiphot microscope (Nikon, Inc., Melville, N.Y.) connected to a charge-coupled device camera. Image analysis software and hardware were used to digitize and process data (MCID: Image Research Inc., St. Catharines, Ontario, 1995). Screen resolution for displaying the digitized images was 1280×1024 pixel in 8-bit monochrome/256 digital gray levels, with calibrated spatial measurement. The target islets were outlined and analyzed according to a set range of relative optical density levels. In each section, either all islets or a minimum of 9-10 randomly selected islets were examined. Islet area, % glucagon positive cells, % insulin positive cells, and insulin/glucagon ratio were determined for each islet.

Serum insulin levels were measured either at baseline, at diabetes onset or at 120 days of age by a conventional radioimmunoassay using rat insulin as a standard. (Immunoassay Core, Diabetes Endocrinology Research Center, University of Washington, Seattle). Serum PAF-AH activity was measured in blood collected at the time of euthanasia by utilizing a radioactive form of PAF substrate, 2-[acetyl-³H] PAF, which reacts with PAF-AH and produces lyso-PAF and [³H] acetate. A sample containing rPAF-AH was serially diluted in assay buffer to ensure that the rPAF-AH enzyme level was within the detectable limit of the assay (61 ng/ml). Along with a control standard, the samples were incubated with the radiolabelled substrate at 37° C. for a set period. From the reaction, lyso-PAF and unreacted 2-[acetyl-³H] PAF were precipitated out. The remaining fraction that contains [³H] acetate was then quantified in a liquid scintillation counter. The enzyme activity of the sample was calculated from the concentration of [³H] Acetate liberated during the incubation.

The Kaplan-Meier estimator was used to obtain survival curves for various groups. Equiality of survival curves was tested with the log-rank or Mantel-Haenzel test. Differences in frequencies were tested by Chi square analysis with Yates correction. Differences in insulitis severity were assessed with the Kruskal Wallis test. In cases where large sample distributions for test statistics were not valid, p-values were based on exact permutational distributions. The Mann-Whitney U test was used to test differences in morphometric analysis, serum insulin, and serum PAF-AH activity because of skewed variables.

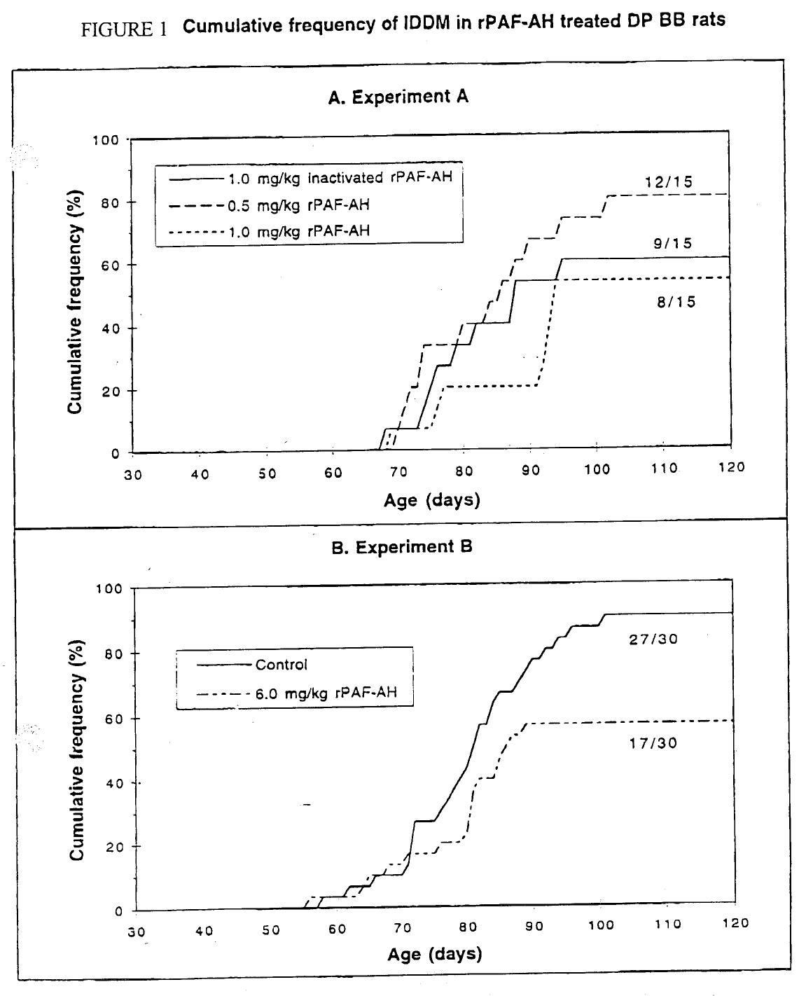

The frequency of diabetes in each treatment group is displayed in FIG. 1A for experiment A and FIG. 1B for experiment B. During the course of experiment A, there was a significant reduction in the frequency of diabetes between the 1.0 mg/kg rPH.2 and 0.5 mg/kg rPH.2 treated group at day 90 (p<0.01). At 120 days of age, the 0.5 mg/kg rPH.2 treated group had 12/15 (80%) diabetic rats; the inactivated rPH.2 treated group had 9/15 (60%) diabetic rats; and the 1.0 mg/kg rPH.2 treated group 8/15 (53%) diabetic rats. The age of onset ranged from 68 to 102 days, with a mean of 86 days for the 1.0 mg/kg rPH.2 treated group, 82 days for the 0.5 mg/kg rPH.2 treated group, and 81 days for the inactivated rPH.2 treated group. The Kaplan-Meier survival analysis, which took delays in age of onset into account, showed no difference between the three groups.

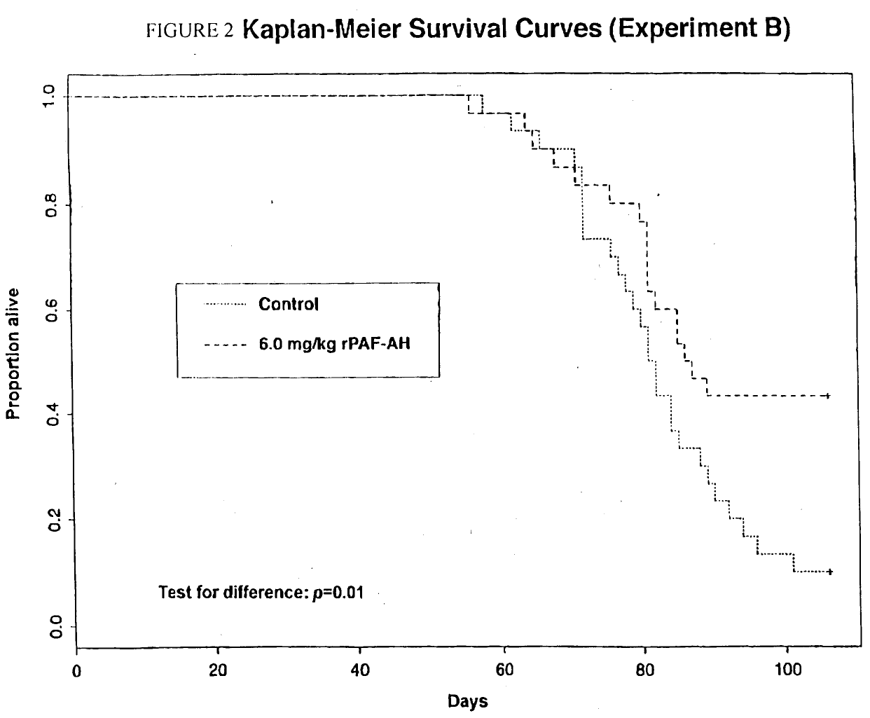

In experiment B, a difference in the frequency of diabetes between the rPH.2 treated and the control group began to emerge at day 90. See FIG. 1B. There was a significant difference in the diabetes frequency at 120 days of age, as indicated by 57% (17/30) in the group treated with 6.0 mg/kg rPH.2 group and 90% (27/30) in the controls (p=0.0044). The age of onset ranged from 56 to 101 days, with a mean of 78 days for the 6.0 mg/kg rPH.2 treated group and 80 days for the control group. The Kaplan-Meier survival analysis, shown in FIG. 2, showed a significant improvement (p=0.01) in the 6.0 mg/kg rPH.2 treated group compared to the controls.

In both experiments A and B, rPH.2 did not affect the overall growth rate within the different treatment groups. All groups displayed a normal growth curve until approximately 1-2 days prior to diabetes onset, when the average weight loss was 4-5 grams. No significant differences among the groups were detected in body weight, blood glucose levels at the day of onset, organ weight (pancreas, spleen, liver kidney, thymus and adrenal gland) or thyroiditis score.

Insulitis scores from experiments A and B are shown respectively in Tables 1A and 1B below. No correlation between the degree of insulitis and the age of onset was observed in either experiment A or B. In experiment A, the mean insulitis score among the diabetic animals ranged from 3.3 to 3.8 compared to scores of 0.67 to 2 for the non-diabetic animals. In experiment B, a significant difference in mean insulitis score was found between the diabetic (2.5±0.9) and non-diabetic rats (0.38±0.5) in the 6.0 mg/kg rPH.2 treated group (p=0.0001). A lower mean insulitis score was also noted in non-diabetic rats in the 6.0 mg/kg rPH.2 treated group (0.38±0.5) compared to the non-diabetic controls (0.67±0.6) (p=0.04).

TABLE 1A

| | 1.0 mg/kg | | |
|---|---|---|---|
| | inactivated | | |
| Experiment A: | rPH.2 | 0.5 mg/kg rPH.2 | 1.0 mg/kg rPH.2 |
| Mean insulitis score | 3.6 ± 0.7 | 3.3 ± 1.0 | 3.8 ± 0.7 |
| for diabetic rats | | | |
| Mean insulitis score | 0.67 ± 0.8 | 1.7 ± 2.1 | 2 ± 1.3 |
| for non-diabetic rats | | | |

TABLE 1B

| Experiment B: | Control | 6.0 mg/kg rPH.2 |
|---|---|---|
| Mean insulitis score for | 2.4 ± 0.9 | 2.5 ± 0.9* |
| diabetic rats | | |
| Mean insulitis score for | 0.67 ± 0.6 | 0.38 ± 0.5 |
| non-diabetic rats | | |

Results of morphometric analysis of pancreatic cells in experiments A and B are shown respectively in Tables 2A and 2B below. In experiment A, morphometric analysis indicated that among rats that developed diabetes, the 1.0 mg/kg rPH.2 treatment group had a larger average islet area than the inactivated rPH.2 treatment group (0.0312±0.0205 mm² and 0.0224±0.0114 mm² respectively). In experiment B, there was no difference in the islet area between the diabetic and non-diabetic animals nor between the different treatment groups. The percentage of insulin positive cells was lower in all diabetic rats while the percentage of glucagon immunoreactive cells was higher in all diabetic rats compared to the non-diabetic rats. This is consistent with the loss of insulin at diabetes onset and in agreement with the assumption that an individual will not display significant IDDM symptoms until about 80% of the β cells have been destroyed.

TABLE 2A

| | | 1.0 mg/kg | | |
|---|---|---|---|---|
| Experiment | | inactivated | 0.5 mg/kg | 1.0 mg/kg |
| A: | Diabetes | rPH.2 | rPH.2 | rPH.2 |
| no. tested | + | 8.6 | 8.9 | 9.5 |
| (islets/rat): | − | 12.3 | 10.0 | 9.0 |
| Area per islet | + | 0.0224 ± 0.0114 | 0.0239 ± 0.0118 | 0.0312 ± 0.0205 |
| (mm²) | − | 0.0301 ± 0.0222 | 0.0340 ± 0.0107 | 0.0247 ± 0.0080 |
| % Glucagon | + | 23.7 ± 7.8 | 24.5 ± 12.0 | 27.5 ± 11.5 |
| cells | − | 20.6 ± 4.2 | 20.1 ± 2.0 | 19.2 ± 3.53 |
| % Insulin | + | 1.9 ± 4.7 | 4.2 ± 11.7 | 0.0 ± 0.0 |
| cells | − | 32.5 ± 17.9 | 18.4 ± 18.9 | 15.0 ± 14.9 |
| Insulin/ | + | 0.2 ± 0.4 | 0.8 ± 1.9 | 0.0 ± 0.0 |
| Glucagon | − | 2.0 ± 1.2 | 1.0 ± 0.9 | 1.1 ± 1.1 |
| ratio | | | | |

TABLE 2B

| Experiment B: | Diabetes | Control | 6.0 mg/kg rPH.2 |
|---|---|---|---|
| no. tested (islets/rat): | + | 9.1 | 9.2 |
| | − | 9.3 | 11.1 |
| Area per islet (mm²) | + | 0.0378 ± 0.0867 | 0.0213 ± 0.0068 |
| | − | 0.0247 ± 0.0116 | 0.0253 ± 0.0097 |
| % Glucagon cells | + | 21.3 ± 10.9 | 18.7 ± 9.9 |
| | − | 11.3 ± 5.9 | 13.4 ± 5.5 |
| % Insulin cells | + | 12.0 ± 10.7 | 11.5 ± 9.7 |
| | − | 28.5 ± 15.7 | 30.5 ± 14.8 |
| Insulin/Glucagon ratio | + | 1.2 ± 1.3 | 1.3 ± 1.6 |
| | − | 3.8 ± 3.0 | 3.7 ± 3.5 |

Serum insulin levels were determined at the time of diabetes onset or at 120 days of age. At the time of diabetes onset in experiment A, the serum insulin levels were highly variable. In the non-diabetic rats euthanized at 120 days, the 1.0 mg/kg rPH.2 treated group had higher serum insulin levels (10.7±4.5 μU/ml) compared to the 0.5 mg/kg rPH.2 treated group (6.8±1.1 μU/ml) and the group treated with inactivated rPH.2 (6.1±0.5 μU/ml) (p=0.0012). In experiment B, the serum insulin levels of the diabetic animals were 5- to 10-fold higher than the non-diabetic rats and all animals in experiment A. The serum insulin levels of the diabetic animals treated with 6.0 mg/kg rPH.2 were approximately doubled (101.9±144.2 μU/ml) compared to the diabetic controls (54.5 ±101.4 μU/ml) (p=0.2137) and 10 times higher than the non-diabetic 6.0 mg/kg rPH.2 treated group (10.5±5.6 μU/ml) (p=0.0019). There was no difference between the treatment groups in the non-diabetic animals.

PAF-AH enzyme activity was tested in blood sample collected (without morning injections for experiment A and within 8 hours of the injection for experiment B) at onset of diabetes or at the end of the injection period (120 days of age) for both series of experiments. In experiment A, diabetic rats that were treated with 1.0 mg/kg or 0.5 mg/kg rPH.2 exhibited a 4.2- and 2.2-fold increase in PAF-AH activity, respectively, compared to the diabetic rats treated with inactivated rPH.2. For the non-diabetic animals, the 0.5 mg/kg rPH.2 treated group demonstrated a 4-5 fold increase in PAF-AH activity over the 1.0 mg/kg rPH.2 treated group (p=0.0264) and the inactivated rPH.2 treated group (p=0.0162). There was no difference in PAF-AH activity between the 1.0 mg/kg rPH.2 treated group and the inactivated rPH.2 treated group. In experiment B, the i.p. injections of 6.0 mg/kg rPH.2 resulted in a 18- to 27-fold increase in endogenous levels of rPH.2 compared to the controls regardless of whether the rats developed diabetes or not (p=0.0001 and p=0.0036, respectively).

It is remarkable that a systemically active agent, rPAF-AH product, which modulates the levels of PAF circulating in vivo, was able to reduce the frequency of diabetes and preserve islet β cells in diabetes prone BB rats. The best effect was observed in experiment B with 6.0 mg/kg rPH.2 injected daily to maintain high serum levels of rPH.2, which reduced the incidence of diabetes from 90% to 57% and provided protection from insulitis. rPAF-AH products may affect diabetes by reducing the levels of PAF in the islets of Langerhans and thereby inhibiting the inflammatory process that results in destruction of β cells, possibly through an inhibition of macrophages or a reduction in the rate at which autoaggressive T and B cells infiltrate the pancreas.

The reduction in frequency of diabetes seen in inactivated rPH.2 treated rats may be due to some residual activity of the enzyme. Since rPAF-AH has a half-life of 8 hours in rats, the lack of protection from the 0.5 mg/kg rPH.2 treatment could be attributed to inadequate levels of circulating rPAF-AH in the interval between treatments. Recombinant PAF-AH has a half-life in rats of about eight hours when administered intravenously. The data in both series of experiments showed that PAF-AH activity is still detected six to eight hours afer the last intraperitoneal injection of rPH.2. Further experiments aimed at maintaining a high plasma rPAF-AH activity may identify optimal therapeutic dose levels.

Numerous modifications and variations in the practice of the invention are expected to occur to those skilled in the art upon consideration of the foregoing description of the presently preferred embodiments thereof Consequently, the only limitations which should be placed upon the scope of the present invention are those which appear in the appended claims.

What is claimed is:

1. A method for preventing diabetes mellitus comprising the step of administering to a subject at risk of developing diabetes mellitus an amount of a PAF-AH product effective to prevent diabetes mellitus.

2. The method of claim 1 wherein the PAF-AH product is rPH.2 or rPH.9.

3. The method of claim 1 wherein the amount of PAF-AH product administered ranges from about 1 μg/kg to about 100 mg/kg daily.

4. The method of claim 1 wherein the subject has signs of insulitis.

5. The method of claim 1 wherein the subject has elevated levels of anti-islet cell antibodies.

6. The method of claim 5 wherein the anti-islet cell antibodies are selected from the group consisting of anti-insulin antibodies, anti-GAD (glutamic acid decarboxylase) antibodies and anti-islet antigen₂ antibodies.

7. A method of slowing the progression of diabetes mellitus comprising the step of administering to a subject with diabetes mellitus a PAF-AH product in an amount effective to prevent further destruction of insulin-secreting pancreatic islet cells.

8. The method of claim 7 wherein the subject is suffering from insulin dependent diabetes mellitus.

9. The method of claim 7 wherein the PAF-AH product is rPH.2 or rPH.9.

10. The method of claim 7 wherein the amount of PAF-AH product administered ranges from about 1 μg/kg to about 100 mg/kg daily.
